(19) United States
(12) Patent Application Publication
(10) Pub. No.: US 2019/0262347 A1
(43) Pub. Date: Aug. 29, 2019

(54) COMPOSITION COMPRISING COMBINATION OF EPICATECHIN AND ANTI-CANCER COMPOUND

(71) Applicant: SPHAERA PHARMA PVT. LTD., Haryana (IN)

(72) Inventor: Sundeep DUGAR, Haryana (IN)

(73) Assignee: SPHAERA PHARMA PVT. LTD., Haryana (IN)

(21) Appl. No.: 16/345,790

(22) PCT Filed: Nov. 1, 2017
(86) PCT No.: PCT/IN2017/050506

§ 371 Date: Apr. 29, 2019

(30) Foreign Application Priority Data
Nov. 1, 2016 (IN) 201611037375

(51) Int. Cl. A61K 31/5355; A61K 31/353; A61P 35/00

(52) U.S. Cl. CPC A61K 31/5355; A61P 35/00; A61K 31/353

(57) ABSTRACT

The present invention discloses a novel, stable and synergistic combination of epicatechin with anti-cancer compounds. The present invention also discloses a composition comprising the novel combination of epicatechin with anti-cancer compounds along with other pharmaceutically acceptable excipients.

### FIELD OF THE INVENTION

The present invention is drawn to a novel combination of epicatechin with anti-cancer compounds and a composition comprising the novel combination.

### BACKGROUND OF THE INVENTION

Cancer is one of the most prevalent disease in humans and accounts for most of the mortality and morbidity in humans resulting in death of millions of people annually and also diminishing the quality of life of a patient. Cancer as a group of disease has the hallmark of abnormal cell growth with the potential to invade and/or spread to other parts of the body and incidences of all cancers are on the rise all over the world. Incidences of cancers are generally associated with, amongst other things, genetic factors, exposure to particular toxins and known cancer causing substances, diet, habits such as smoking (tobacco). Changes in the genetic and metabolic pathways within the cancer cell have been established as drivers of the disease. The Warburg effect is the observation that most cancer cells predominantly produce energy through glycolysis followed by lactic acid fermentation rather than oxidation of pyruvate in mitochondria as in most normal cells. (Gatenby R A; Gillies R J, Nature Reviews Cancer 4 (11): 891-9, 2004; Kim J W, Dang C V, Cancer Res. 66 (18): 8927-8930, 2006). The latter process is aerobic (uses oxygen). Malignant, rapidly growing tumor cells typically have glycolytic rates up to 200 times higher than those of their normal tissues of origin; this occurs even if oxygen is plentiful. Otto Warburg postulated this change in metabolism is fundamental to cancer cells [Warburg O, Science 123 (3191): 309-314, 1956], a claim now known as the Warburg effect. The Warburg effect may simply be a consequence of damage to the mitochondria in cancer, or an adaptation to low-oxygen environments within tumors, or a result of cancer genes shutting down the mitochondria because they are involved in the cell's apoptosis program which would otherwise kill cancerous cells. It may also be an effect associated with cell proliferation. Since glycolysis provides most of the building blocks required for cell proliferation, cancer cells have been proposed to need to activate glycolysis to proliferate. Today, mutations in oncogenes and tumor suppressor genes are thought to be responsible for malignant transformation, and the Warburg effect is considered to be a result of these mutations rather than a cause. [Bertram J S, Mol. Aspects Med. 21 (6): 167-223, 2000.Grandér D, Med. Oncol. 15 (1): 20-26, 1998]. Obesity in conjunction is also a driver of oncogenesis (Oncogene. 2016 Dec. 8; 35(49): 6271-6280). Compounds that inhibit glycolysis are currently the subject of intense research as anticancer agents, [Pelicano H, Martin D S, Xu RH, Huang P Oncogene 25 (34): 4633-4646, 2006] including SB-204990, 2-deoxy-D-glucose (2DG), 3-bromopyruvate (3-BrPA, bromopyruvic acid, or bromopyruvate), 3-BrOP, 5-thioglucose and dichloroacetic acid (DCA). Alpha-cyano-4-hydroxycinnamic acid, a small-molecule inhibitor of monocarboxylate transporters (MCTs; which prevent lactic acid build up in tumors) has been successfully used as a metabolic target in brain tumor pre-clinical research. Dichloroacetic acid (DCA), a small-molecule inhibitor of mitochondrial pyruvate dehydrogenase kinase, “downregulates” glycolysis in vitro and in vivo and might have therapeutic benefits against many types of cancers. Mutations in oncogenes and tumor suppressor genes are also responsible for malignant transformation. Another possibility is to affect the glycolytic pathways in cancer cells is to enhance the mitochondrial pathway and promote oxidative phosphorylation.

Hence, there is a fundamental change in cancer cells that is both metabolic and mitochondrial. Hence, intervention of both the metabolic pathways/mitochondrial pathways and the oncogenic pathways within a cancer cell should have enhanced merit.

Additionally, drugs and compositions of drugs for the treatment of cancer are commonly available to patients, such drugs and compositions are often drawn to a very high dose and long duration of treatment result in various side effects to the patients and also several of these drugs become ineffective due to development of resistance. Hence reduction of dose of these drugs and duration of treatment will provide a significant benefit to the patients by reducing the side effects while enhancing the efficacy.

Flavonols present in chocolate, tea, fruits, vegetables and wine have been reported for their use in the treatment of cancer due to their antioxidant activity. For example: catechins have previously been reported to enhance the effect of the anti-cancer compounds, e.g., Adriamycin and doxorubicin (Sugiyama and Sadzuka, 1998, Can. Lett. 133:19-26 and Sadzuka et al., 1998, Clin. Can. Res. 4:153-156). But often the flavanols do not affect the metabolic and mitochondrial pathway. Initial research has demonstrated that epicatechin is effective in enhancing the metabolic and mitochondrial pathway and that this activity was significantly better than other flavanols, and in particular specific to (−)-epicatechin and (+)-epicatechin (collectively “epicatechin”) (see PCT/US2012/040929).

Hence, the present application examines the effect of epicatechin with anti-cancer compounds.

### OBJECT OF THE INVENTION

The object of the present invention is to provide a novel, stable and synergistic combination of epicatechin with anti-cancer compounds and a composition comprising the novel combination.

### SUMMARY OF THE INVENTION

The present invention discloses a novel, stable and synergistic combination of epicatechin with anti-cancer compounds. The present invention also discloses a composition comprising the novel combination of epicatechin with anti-cancer compounds along with other pharmaceutically acceptable excipients.

### BRIEF DESCRIPTION OF DRAWINGS

FIGS. 1a depicts the synergistic effect of racemic epicatechin when combined with PI3K/mTOR inhibitor Compounds No. 1004 in colon cancer based on HCT116 cell line induced Xenograft model in mice (oral dosing);

FIG. 1b depicts the synergistic effect of racemic epicatechin when combined with PI3K/mTOR inhibitor Compounds No. 1004 in reduction of the tumor weight;

FIG. 2a depicts the effect of cisplatin in inhibition of cell growth;

FIG. 2b depicts the effect of epicatechin in inhibition of A549;

FIG. 2c depicts the principles involved in isobologram;

FIG. 2d depicts an isolbologram demonstrating the synergistic effect of cisplatin and (−) epicatechin;

FIG. 3 depicts the synergistic effect of (−) epicatechin and cisplatin in cancer cell lines such as NCI-H1299 and HCC-827; and

FIGS. 4a and 4b depict the synergistic effect of (−) epicatechin and cisplatin in apoptosis.

### DETAILED DESCRIPTION OF THE INVENTION

The present invention discloses a novel anti-cancer combination of epicatechin with at least one anti-cancer compound.

The epicatechin of the present invention may be selected from the group comprising, (+)-epicatechin, (−)-epicatechin or mixtures of (+)-epicatechin and (−)-epicatechin.

The epicatechin may be present in a ratio varies in the range from 0.1% to 99.9% to 99.9% to 0.1% of the combination of the present invention, and the remaining component of the combination may be an anticancer compound. The present invention discloses a novel, stable and synergistic combination of pure isomers of epicatechin, mixtures of epicatechin with anti-cancer compounds. (+)-epicatechin: (−)-epicatechin may be present in a ratio varies i in the range of 0.1:99.9 to 99.9:0.1.

The epicatechin of the present invention may be obtained from natural or synthetic sources.

The anti-cancer compound of the present invention may be selected from the group comprising alkylating antineoplastic compounds such as cyclophosphamides, nitosoureas, alcohol sulfonates; Platinum coordination compounds such as cisplatin, carboplatin, oxaliplatin; antimetabolites such as methotrexate, 6-mercaptopurine and 5-fluorouracil (5-FU), Gemcitabine; anti-tumor antibiotics such as doxorubicin; microtubule inhibitors like docetaxel, paclitaxel, topotecan, etoposide, irinotecan, vinblastine; biological compounds such as imatinib, lapatinib, sunitinib, sorafenib, temsirolimus; bisphosphonates such as ibandronic acid, zolendronic acid immunotherapeutic compounds; targeted anti-cancer therapeutic compounds and other general chemotherapeutic compound such as the group comprising selective or non-selective PI3Kinase inhibitors, mTOR inhibitors, MEK inhibitors, Akt inhibitors, tyrosine kinase inhibitors such as imatinib, erlotinib and gefitinib aiming at EGF receptor; sunitinib inhibitor for FGF, VEGF, PDGF; ALK inhibitors, ABL, SCR, FLT3, KIT, MET inhibitors, BRAF inhibitors, IIβinhibitors, JAK1/2, JAK 3 inhibitors, proteosome inhibitor Bortezomib, other growth factor inhibitors, inhibitors of RAS/RAF/MAPK pathway and other signal-transduction inhibitors, multi-targeted kinase inhibitors, topoisomerase inhibitors, glycolytic inhibitors, cathepsin B inhibitors, histone deacetylase inhibitors and the same and may be used either individually or in combination and other anti-cancer compound as known to those skilled in the art.

Preferably, the anticancer compound of the present invention may be selected from group comprising platinum-containing anti-cancer drugs such as cisplatin, carboplatin or oxaliplatin, chemotherapeutic compounds such as PI3kinase/mTOR inhibitors.

The anti-cancer compounds may be present in a ratio from 0.01 to 99.99 based on the novel combination of the present invention.

The anti-cancer compound of the present invention may be a PI3Kinase/mTORinhibitor as listed herein below at Table 1 or may be selected from other compounds that possess PI3Kinase/mTOR.

TABLE 1
Illustrative compounds having PI3K/mTOR activity.

| Compound | | |
|---|---|---|
| Number | Structure | IUPAC name |
| 1001 | 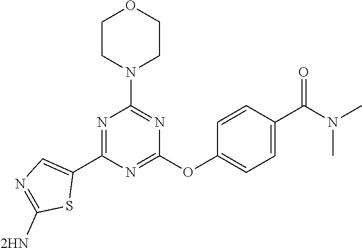 | 4-(4-(2-aminothiazol-5- yl)-6-morpholino-1,3,5- triazin-2-yloxy)-N,N- dimethylbenzamide |
| 1002 | 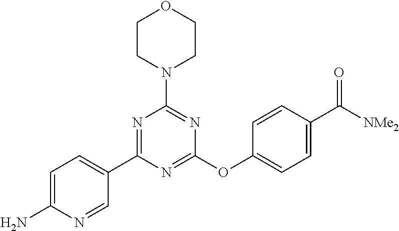 [SMILES: CN(C)C(=O)c1ccc(Oc2nc(-c3ccc(N)nc3)nc(N3CCOCC3)n2)cc1] | 4-(4-(6-aminopyridin-3- yl)-6-morpholino-1,3,5- triazin-2-yloxy)-N,N- dimethylbenzamide |
| 1003 | 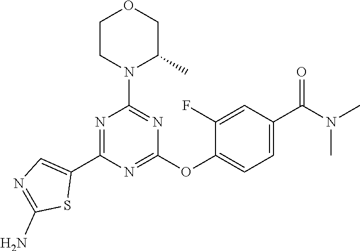 [SMILES: C[C@H]1COCCN1c1nc(Oc2ccc(C(=O)N(C)C)cc2F)nc(-c2cnc(N)s2)n1] | (S)-4-((4-(2-aminothiazol- 5-yl)-6-(3- methylmorpholino)-1,3,5- triazin-2-yl)oxy)-3-fluoro- N,N-dimethylbenzamide |
| 1004 | 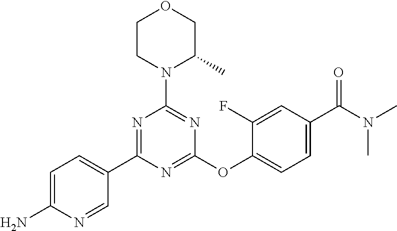 [SMILES: C[C@H]1COCCN1c1nc(Oc2ccc(C(=O)N(C)C)cc2F)nc(-c2ccc(N)nc2)n1] | (S)-4-((4-(6-aminopyridin- 3-yl)-6-(3- methylmorpholino)-1,3,5- triazin-2-yl)oxy)-3-fluoro- N,N-dimethylbenzamide |

In another aspect, the present invention discloses a composition comprising the novel combination of the present invention along with other pharmaceutically acceptable excipients.

The composition of the present invention may be formulated in a manner suitable for administration in oral, topical, or parenteral dosage form.

Without being limited by theory, it is submitted that the present invention discloses a novel combination of epicatechin and an anti-cancer compounds acts synergistically and substantially enhances the effect in alleviating in various cancers, synergistic effect in treatment of cancer, reduces the risk of developing resistance of patients towards anti-cancer combination, reducing effects associated with obesity, inducing apoptosis in cancer cell lines, inducing immune response for cancer cells, reducing Warburg effect as illustrated in examples 1-3.

### ADVANTAGES

1. The combination of the present invention is novel and has decreased side effects and increased efficacy.

2. The combination of the present invention is stable and has synergistic effect.

The following examples further illustrate the invention and its unique characteristics in elaborate manner. However the following examples are not intended to limit the scope of the invention in any way.

### EXAMPLE 1

### Evaluation of the Synergistic Effect of Epicatechin in Combination with PI3K/mTOR Inhibitor, Compound 1004

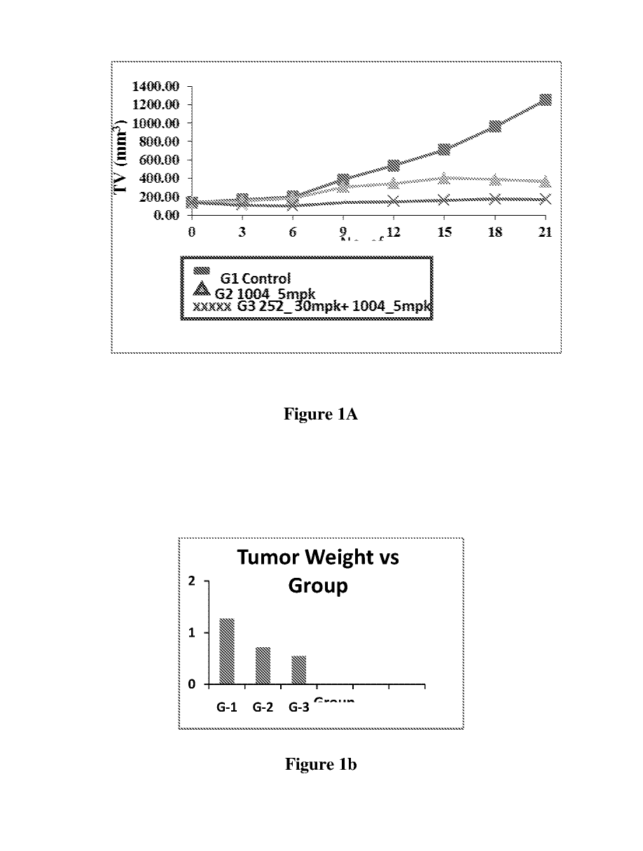

Anticancer potential of epicatechin in combination with a PI3K/mTOR inhibitor is evaluated against cancer xenograft model in immunocompromised mice. CD1 nude mice are dosed for a period of 21 days with vehicle control, PI3K/mTOR inhibitor and a combination of PI3K/mTORinhibitor and epicatechin. The reduction in tumor volume is found to be maximum in the group (G-3) with a tumor growth inhibition % (TGI %) of 97% when doses in combination. The results are presented at Table 2, Table 3 and FIGS. 1a and 1b.

TABLE 2
Results of combination of epicatechin and compound
No. 1004 in HCT116 induced xenograft model in mice

| 21daysefficacy study in HCT116 | | | |
|---|---|---|---|
| induced xenograft model in mice | | TGI % (Mean) | T/C % (Mean) |
| G-1 | CONTROL | 0 | 0 |
| G-2 | 1004_5 MPK | 80 | 29 |
| G-3 | Epicatechin_30 mpk + | 97 | 14 |
| | 1004_5 mpk | | |

TGI: Tumor growth inhibition
T/C: Treated/Control on 21^{st} day

TABLE 3
Results of combination of epicatechin and compound
No. 1004 in reduction of the tumor weight

| Group | Tumor weight (g) |
|---|---|
| Control | 1.27 |
| 1004 | 0.72 |
| Epicatechin _30 mpk + 1004_5 mpk | 0.55 |

From the data present at Table 2 and Table 3 and FIGS. 1a and FIG. 1b, it can be seen that the combination of epicatechin and compound 1004 acts synergistically.

### EXAMPLE 2

### Evaluation of the Synergistic Effect of (−) Epicatechin in Combination with Cisplatin

2.1 Cell culture: Hell-299 cell line, corresponding to normal lung cells is used as normal cells control and A549 Cell line corresponding to lung adenocarcinoma are cultured under standard conditions under 5% CO₂ at 37° C. Cells are treated with different concentrations of cisplatin [CDDP (cis-Diammine-platinum (ii) dichloride, Sigma)] [1-100 μM] or (−)-epicatechin (EC, Sigma) [0.1-10000 μM] or the combination of both compounds for 48 hours. Both compounds are dissolved in DMSO (0.9%).

2.2 Cell viability: Cell viability is determined by MTT assay. Briefly, cell are incubated with 0.1mg/ml MTT (3-(4,5-dimethyl-2-thiazolyl)-2,5-diphenyltetrazolium bromide) during 40 minutes at 37° C. Purple formazan is solubilized using 0.01M HCl-Isopropanol. The dissolved material is measured spectrophotometrically at 595 nm (BioteckSynergy HT).

Percent viability is calculated as follows:

$$Percent viabilit y \text{:} \frac{optical density of t he experimental group}{optical density of the control group} \times 100$$

Isobolographic Analyses: After determining the concentration-response curves for EC and CDDP, an isobolographic analysis is conducted. This method allows first a theoretical analysis effects of dose combinations, is based on the work reported by Tallarida which evaluates quantitatively and graphically the type of interaction between any two drugs. Briefly, after the inhibitory concentrations (IC) for each compound are calculated, theoretical values (e.g. IC₅₀, IC₃₀ and IC₁₅) of combinations in a fixed ratio 1:1 are obtained according to equation (Eq. (1)) then they get substituted by experimental values (Eq. (2)).

$$\frac{CDDP Theoretical}{ICx} + \frac{EC Theoretical}{ICx} = 1 \qquad Equation ( 1 )$$

Meaning that, to determine if an additive effect exists 1/2 EC effective concentration plus 1/2 CDDP effective concentration must be equal to 1 (one). As example, in a combination as follow 1/2 EC (IC₃₀)+1/2 CDDP (IC₃₀), if an additive effect exists then there will be 30% of inhibition in experimental conditions.

The interaction of EC with CDDP is then experimentally evaluated by the simultaneous administration of 1/2 EC (ICₓ)+1/2 CDDP (ICₓ) concentrations, where ICx correspond to different concentrations but always in 1:1 ratio. The experimental results obtained with the combinations is employed and allowed determination of the type of interaction observed between the two compounds:

$$\frac{{CDDP}^{′} Experimental}{IC 30} + \frac{{EC}^{′} Experimental}{IC 30} = Result \qquad Equation ( 2 )$$

As mentioned, when the experiment produces a result equal to 1, there is an additive effect. If the result is <1, there is a synergism or supradditive effect and if the result is >1 the effect is antagonistic.

Apoptosis Analysis (Acridine Orange/Ethidiumbromide Dyeing)

Presence of apoptosis is evaluated using acridine orange/ethidiumbromidedyeing [15 mM/0.002 mM]. Acridine orange(AO) dyes nuclei in green. Ethidiumbromide(EB) dyes cellular nucleiin red only when plasmalemma integrity is lost. For image acquisition Epifluorescencemicroscope(Nikon Elipse E600) is used. Alive and in-good condition cells present a glossy green dyeing. Cells in apoptotic process and death cells, shows a glossy red dyeing.

Green and red fluorescent intensity is evaluated using ImageJsoftware(version 1.38x http://rsb.info.nih.gov/ij)

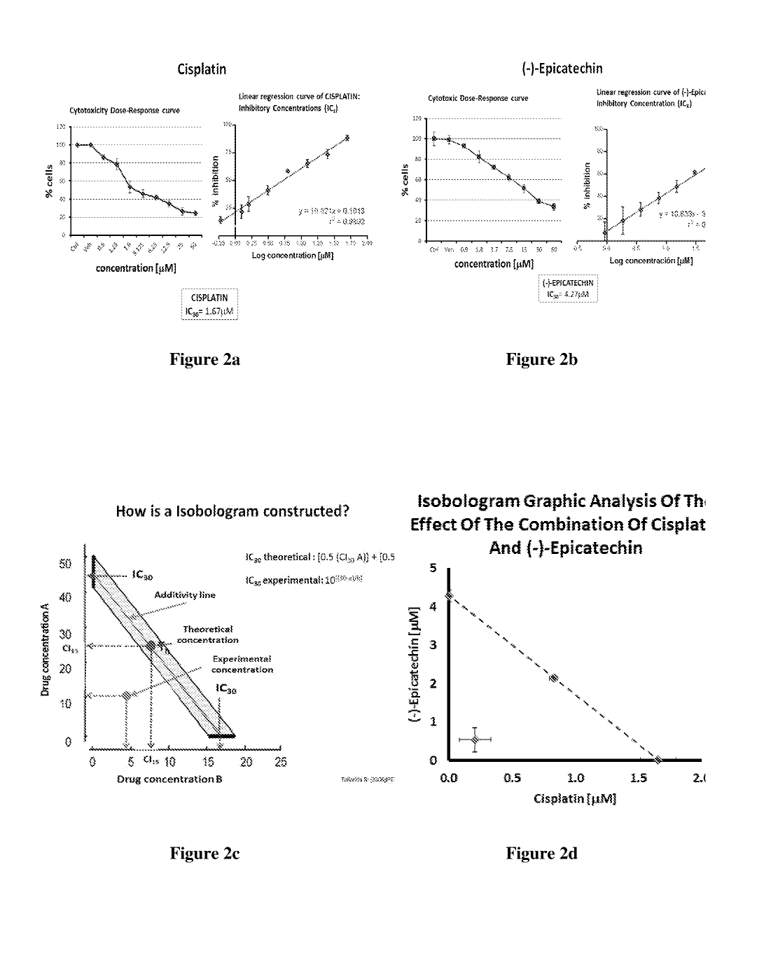

The synergistic activity of epicatechin with cisplatin in reducing cytotoxicity is evaluated in A549 cell lines. FIG. 2a shows the cytotoxic effect of cisplatin and FIG. 2b shows the effect of cytotoxic effect of epicatechin in A549 cells. The effect of the combination of the present invention is represented by isobolograms. The construction and interpretation of isobologram is presented at FIG. 2c for ready reference. The isobologram of the combination of the present invention is presented at FIG. 2d.The red dot located below the line of additivity represents the experimental combination of both compounds needed to achieve 30% of effects (cytotoxicity).The necessary concentrations of (−)-epicatechin and cisplatin to achieve a 30% inhibition of cell growth, when combined, are much lower than the necessary doses to achieve the same level of effect when they are applied separately. Mathematical analysis is done to determine the combination index (γ)

$$γ = \frac{{IC}_{30} experimental}{{IC}_{30} theoretical} = \frac{0.741}{2.96} = 2.50$$

$$\begin{matrix}γ = 1 & ADDITION \\ γ < 1 & SYNERGISM \\ γ > 1 & ANTAGONISM\end{matrix}$$

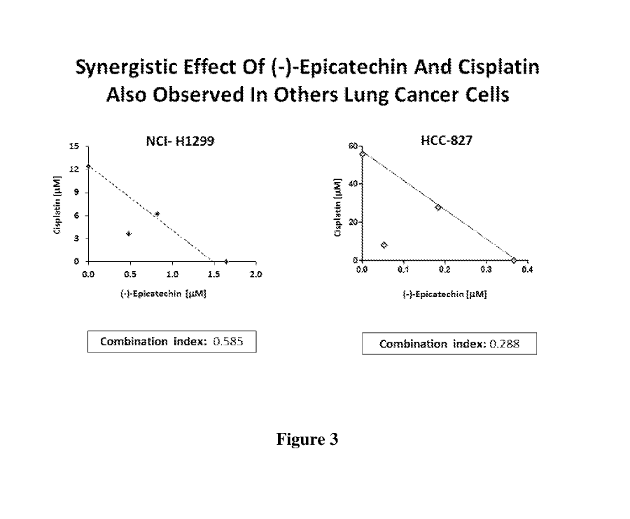

Results: From FIG. 3, it may be discerned that the combination of (−) epicatechin and cisplatin, according to present invention is synergistic in cytotoxic models based on lung cancer cell lines such as NCI-HI299 ad HCC-827.

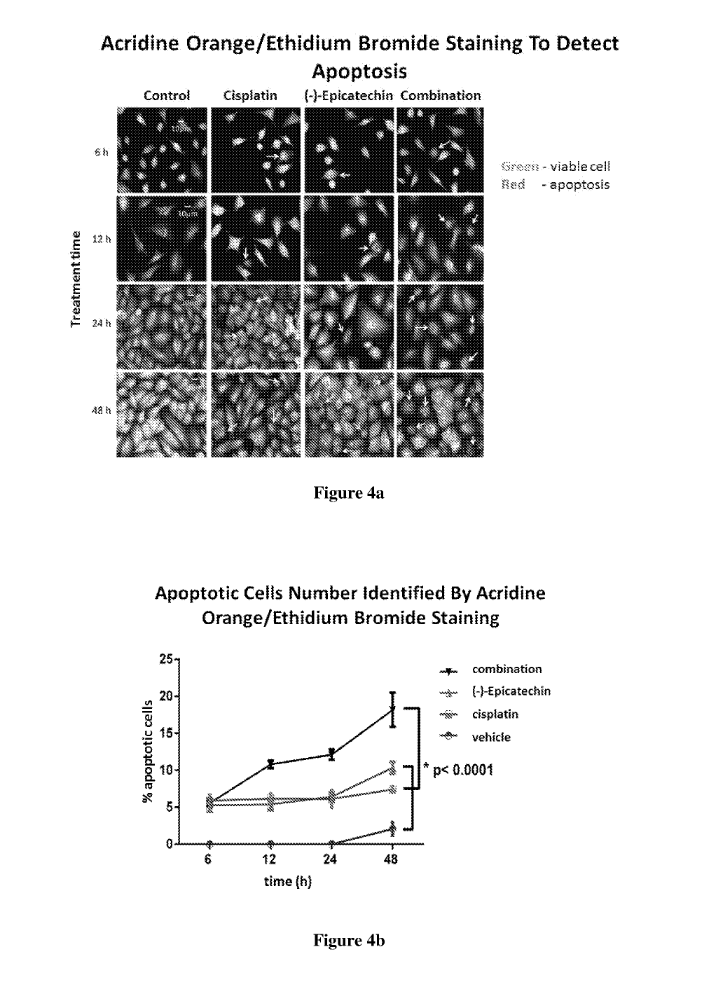

From FIG. 4, it may be discerned that the combination of (−) epicatechin and cisplatin has synergistic effect in inducing apoptosis in cancer cell lines in comparison to the apoptotic effect of (−) epicatechin and cisplatin when these are tested individually.

1-13. (canceled)

14. A composition comprising epicatechin and at least one anti-cancer compound.

15. The composition of claim 14, wherein the epicatechin is selected from (+)-epicatechin, (−)-epicatechin, and mixtures of (+)-epicatechin and (−)-epicatechin.

16. The composition of claim 14, wherein (+)-epicatechin : (−)-epicatechin ratio is in a range of 0.1:99.9 to 99.9:0.1.

17. The composition of claim 14, wherein the epicatechin: anti-cancer ratio is in a range of 0.1:99.9 to 99.9:0.1.

18. The composition of claim 14, wherein the anti-cancer compound is selected from alkylating antineoplastic compounds, platinum coordination compounds, antimetabolites, anti-tumor antibiotics, microtubule inhibitors, biologicals, bisphosphonates, immunotherapeutic compounds, targeted anti-cancer therapeutic compounds, selective or non-selective PI3Kinase inhibitors, mTOR inhibitors, MEK inhibitors, Akt inhibitors, tyrosine kinase inhibitors, inhibitors of FGF, VEGF, and PDGF, ALK inhibitors, ABL, SCR, FLT3, KIT, and MET inhibitors, BRAF inhibitors, IIβ inhibitors, JAK1/2 inhibitors, JAK 3 inhibitors, proteosome inhibitors, growth factor inhibitors, inhibitors of RAS/RAF/MAPK pathway, signal-transduction inhibitors, multi-targeted kinase inhibitors, topoisomerase inhibitors, glycolytic inhibitors, cathepsin B inhibitors, histone deacetylase inhibitors, and any combinations thereof.

19. The composition of claim 18, wherein the anti-cancer compound is selected from cyclophosphamides, nitosoureas, alcohol sulfonates, cisplatin, carboplatin, oxaliplatin, methotrexate, 6-mercaptopurine, 5-fluorouracil (5-FU), gemcitabine, doxorubicin, docetaxel, paclitaxel, topotecan, etoposide, irinotecan, vinblastine, imatinib, lapatinib, sunitinib, sorafenib, temsirolimus, ibandronic acid, zolendronic acid, erlotinib, gefitinib, Bortezomib, and combinations thereof.

20. The composition of claim 18, wherein the anticancer compound is selected from cisplatin, carboplatin, oxaliplatin, and PI3Kinase/mTOR inhibitors.

21. The composition of claim 14, wherein the anti-cancer compound is a PI3Kinase/mTOR inhibitor and is selected from:
i. 4-(4-(2-aminothiazol-5-yl)-6-morpholino-1,3,5-triazin-2-yloxy)-N,Ndimethylbenzamide;
ii. 4-(4-(2-aminothiazol-5-yl)-6-morpholino-1,3,5-triazin-2-yloxy)-N,Ndimethylbenzamide;
iii. (S)-4-((4-(2-aminothiazol-5-yl)-6-(3-methylmorpholino)-1,3,5-triazin-2-yl)oxy)-3-fluoro-N,N-dimethylbenzamide; and
iv. (S)-4-((4-(6-aminopyridin-3-yl)-6-(3-methylmorpholino)-1,3,5-triazin-2-yl)oxy)-3-fluoro-N,N-dimethylbenzamide.

22. The composition of claim 14, wherein the anti-cancer compound that possesses PI3Kinase/mTOR activity has one of the following formulas:

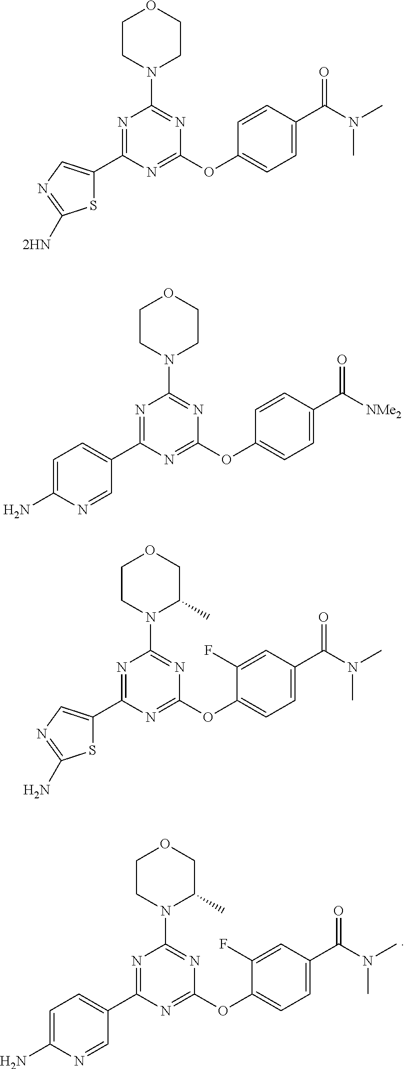

23. The composition of claim 18, wherein the anti-cancer compound that possesses PI3Kinase/mTOR activity has one of the following formulas:

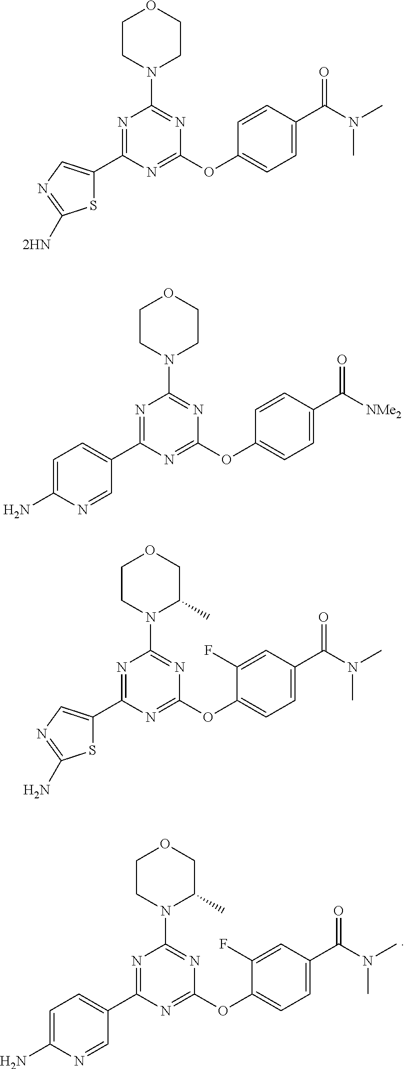

24. A composition comprising the composition of claim 14 and a pharmaceutically acceptable excipient.

25. A method of treating a patient for cancer comprising administering an effective amount of a composition of claim 14.

26. A method of reducing the risk of developing resistance to an anti-cancer composition in a cancer patient, the method comprising administering to the patient an effective amount of a composition of claim 14.

27. A method of reducing effects associated with obesity in a patient, the method comprising administering to the patient an effective amount of a composition of claim 14.

28. A method of inducing immune response for cancer cells in a patient comprising administering to the patient an effective amount of a composition of claim 14.

29. A method of reducing Warburg effect in a cancer patient comprising administering to the patient an effective amount of a composition of claim 14.
